(19) United States
(12) Patent Application Publication
(10) Pub. No.: US 2017/0143348 A1
(43) Pub. Date: May 25, 2017

(54) METHODS AND SYSTEMS FOR THERMOEMBOLIZATION

(71) Applicant: Regents of the University of Minnesota, Minneapolis, MN (US)

(72) Inventor: Erik N.K. Cressman, Missouri City, TX (US)

(21) Appl. No.: 15/360,376

(22) Filed: Nov. 23, 2016

Related U.S. Application Data
(60) Provisional application No. 62/258,829, filed on Nov. 23, 2015.

(51) Int. Cl. A61B 17/12; A61L 24/02; A61L 24/00; A61M 25/00

(52) U.S. Cl. CPC A61B 17/12186; A61B 17/12109; A61M 25/00; A61L 24/02; A61L 24/001; A61L 24/0005; A61M 5/178

(57) ABSTRACT

Thermoembolization systems and techniques may provide simultaneous embolization and ablation of bodily tissue. In some embodiments, thermoembolization may be induced, for example, by arterially infusing a viscous liquid carrying an electrophilic material into the feeding vessel(s) of a tumor. The viscous liquid serves to occlude the arterial supply to the tumor while the electrophilic material concurrently undergoes an exothermic reaction when it comes into contact with water present in bodily tissues. In result, thermoembolization induces simultaneous hypoxic and thermal stresses to the tumor, thereby overwhelming tumor cells.

### CROSS REFERENCE TO RELATED APPLICATIONS

This application claims the benefit of U.S. Provisional Application No. 62/258,829, filed Nov. 23, 2015. The disclosure of the prior application is considered part of and is incorporated by reference in the disclosure of this application.

### TECHNICAL FIELD

This document relates to medical systems and methods for treating tumors and other targeted tissue in the body, and in some embodiments, for transarterial infusion of embolic chemical reagents that induce thermoembolization.

### BACKGROUND

A number of ablation treatments have been used to treat tumors and other tissue in the body. In some cases, for example, ablation therapy may be used to treat tumors (e.g., tumors that are not responsive to chemotherapy or other treatment techniques). An example is primary liver cancer or hepatocellular carcinoma (HCC), which is an aggressive neoplasm that may not respond well to intravenous chemotherapy.

The choice of treatment for cancers such as HCC normally depends on severity of underlying liver disease, size and number of lesions, location of lesions, ability to detect them with MRI, non-contrast or contrast CT, or ultrasound, and local expertise. Conventionally, physicians have targeted tumor tissue with heat by radiofrequency (RF) ablation, microwave ablation, or combined heating with coadministration of drug-containing liposomes, used cryoablation to freeze tumor tissue, or used hepatic arterial drug infusion, bland arterial embolization, chemotherapy combined with arterial embolization, selective internal radioembolization using radioactive labeled iodized oil or radioactive microspheres as the embolic agent, external beam radiation therapy, or direct injection of a single agent (e.g., ethanol, acetic acid, hydrochloric acid, hot saline, or sodium hydroxide) to ablate tumor tissue.

### SUMMARY

Some thermoembolization techniques described herein may provide minimally invasive treatments to solid tumors such as liver cancer, lung cancer, renal cancer, breast cancer, prostate cancer, bone cancer, sarcomas, metastatic disease, or the like. In some embodiments, thermoembolization may be induced, for example, by arterially infusing a viscous liquid carrying an electrophilic material into the feeding vessel(s) of a tumor. The viscous liquid serves to occlude the arterial supply to the tumor while the electrophilic material undergoes hydration when it comes into contact with water (e.g., water present in bodily tissues, or added water or aqueous solutions) such that heat is released as a result of an exothermic reaction. In result, thermoembolization induces truly simultaneous thermal and hypoxic stresses to the tumor, thereby overwhelming tumor cells quickly.

In addition to simultaneous ischemia (hypoxia) and thermal stress, changes in pH of tissue may also be induced. The change in pH also can cause protein denaturation (leading to ablation by itself), and, depending on the material used, can either lower the pH (acidic, as in the acid chloride examples described herein) or raise the pH (alkaline or basic, such as with calcium oxide forming calcium hydroxide).

In one implementation, this document features a thermoembolization system for treating a tissue. The thermoembolization system includes a percutaneous liquid delivery catheter defining at least one lumen extending from a proximal portion of the percutaneous liquid delivery catheter to a distal portion of the percutaneous liquid delivery catheter, a liquid dispensing device including a reservoir and a portion that is configured to releasably couple with the proximal portion of the percutaneous liquid delivery cannula such that the liquid dispensing device can inject a liquid from the reservoir into the lumen of the percutaneous liquid delivery catheter, and a thermoembolic liquid contained within the reservoir. The thermoembolic liquid has a viscosity sufficient to cause an occlusion of a microvasculature of the tissue. The thermoembolic material includes a reagent that exothermically reacts with a substance. The distal portion of the percutaneous liquid delivery catheter is configured for insertion into an arterial vessel such that a distal tip of the distal portion of the percutaneous liquid delivery catheter is positionable adjacent the tissue.

Such a thermoembolization system for treating a tissue may optionally include one or more of the following features. The reagent may comprise calcium oxide. The substance may be water. The reagent may comprise sulfuric acid. The thermoembolic liquid may comprise an ethiodized oil such as LIPIODOL®. The liquid dispensing device may be a syringe. The reagent may comprise beads. The beads may comprise calcium oxide. The beads may comprise an inert coating surrounding the calcium oxide. The thermoembolic liquid may be radiopaque.

In another implementation, a method of treating a tumor includes infusing a thermoembolic liquid into an arterial vessel feeding the tumor. The thermoembolic liquid has a viscosity sufficient to cause an occlusion of a microvasculature of the tumor. The thermoembolic liquid includes a reagent that exothermically reacts with a substance to generate heat sufficient to ablate at least a portion of the tumor while the thermoembolic liquid is occluding the microvasculature.

Such a method of treating a tumor may optionally include one or more of the following features. The reagent may comprise calcium oxide or sulfuric acid. The substance may be water present in the tumor. The thermoembolic liquid may comprise an ethiodized oil such as, but not limited to, LIPIODOL®. The reagent may comprise beads. The beads may comprise calcium oxide. The beads may comprise an inert coating surrounding the calcium oxide. The thermoembolic liquid may be radiopaque. The tumor may be located in a human liver. The tumor may be located in at least one of a kidney, a bone, or a prostate.

In another implementation, a system includes a thermoembolic fluid contained within the reservoir of a fluid dispensation device and a thermoembolization delivery device defining at least one lumen configured to receive the thermoembolic fluid from the reservoir and to output at least a portion of the thermoembolic fluid into an arterial vessel proximate a targeted tissue for exothermically reacting with a substance proximate the targeted tissue.

In another implementation, a system includes a means for containing a thermoembolic fluid and a means for inducing simultaneous ischemia and ablation of a tumor by delivery of the thermoembolic fluid to the tumor in a minimally invasive manner.

In another implementation, a method of inducing simultaneous ischemia and ablation of a targeted tissue includes positioning a thermoembolization delivery device through a vasculature so that an output port is proximate to a targeted tissue, and infusing a thermoembolic liquid from the output port and toward the targeted tissue so as to cause an occlusion of one or more branch vessels of the targeted tissue while at least a portion of the thermoembolic liquid exothermically reacts with a substance at the targeted tissue to generate heat sufficient to ablate at least a portion of the targeted tissue.

Some or all of the embodiments described herein may optionally provide one or more of the following advantages. First, the thermoembolization techniques and systems described herein can induce simultaneous ischemia and ablation to solid tumors (e.g., liver cancer, lung cancer, renal cancer, breast cancer, prostate cancer, sarcomas, or the like) in a minimally invasive fashion. Such techniques and systems may be useful, for example, to treat patients who are not surgical candidates due to the nature of the tumors or other intervening factors. Whereas tumor cells may be capable of adapting to ischemia or ablation treatment techniques administered individually, the simultaneous thermal and hypoxic stresses delivered by thermoembolization can overwhelm tumor cells quickly before they have a chance to adapt. Hence, thermoembolization provides a greater treatment efficacy than the sequential delivery of ischemia and ablation techniques individually.

Second, some of the systems and devices described herein may be optionally manufactured without high-cost components such as RF ablation probes or energy source generators/base units. In addition, some optional embodiments may not need cables or connecting tubing that would transgress the sterile procedure field to connect to a base power unit, thereby adding convenience and improved procedural safety for the treating health care professional and the patient.

Third, the thermoembolization techniques and systems described herein may be used to treat larger tumors in a lower number of treatment sessions, thereby adding convenience to the patient.

Fourth, the thermoembolization techniques and systems described herein may employ real-time monitoring using medical imaging systems, such as fluoroscopic imaging devices or CT. Moreover, in some embodiments, the thermoembolization process can be monitored in an MRI setting without the need for specialized (high-cost), MRI-compatible alloys in the delivery device.

Fifth, in some embodiments of the thermoembolization techniques and systems described herein, the reactions can minimize or otherwise reduce gas formation, resulting in a reduced likelihood of any risk of gaseous embolus. Gas so formed could, in some circumstances, form large bubbles blocking blood flow to the brain, heart, kidneys, or gut leading to infarction. Furthermore, too much gas formation could actually cause a vapor- lock condition in the heart, (generally seen in the right heart in the pulmonary outflow tract) disrupting circulation through the entire body.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an illustration of a human liver that includes an example tumor.

FIG. 2 shows the liver of FIG. 1 with the tumor being treated by a thermoembolization technique, in accordance with some embodiments.

FIG. 3 is a computerized tomography (CT) scan showing a cross-sectional view of a kidney that has received an infusion of thermoembolic materials, in accordance with some embodiments.

FIG. 4 is a graph showing a rise in temperature resulting from exposing a solution of dichloroacetyl chloride in diglyme to water.

FIG. 5 is a photograph showing an experimental setup for infusing thermoembolic materials into a porcine kidney.

FIG. 6 is a graph showing the increase in the internal temperature of the porcine kidney of FIG. 5 resulting from the infusion of the thermoembolic material.

FIG. 7 is a fluoroscopic image of a catheterization of a porcine liver via an aorta of the porcine subject.

FIG. 8 is a fluoroscopic image of the porcine liver of FIG. 7 with the tip of the catheter advanced to the hepatic artery and after an injection of contrast dye.

FIG. 9 is a fluoroscopic image of the porcine liver of FIG. 8 with the tip of the catheter advanced to within a branch of the hepatic artery.

FIG. 10 is a fluoroscopic image of the porcine liver of FIG. 9 after an injection of an embolization liquid in accordance with some thermoembolization devices and techniques described herein.

FIG. 11 is an axial CT scan of a porcine subject after an infusion of thermoembolic materials into a liver of the subject.

FIG. 12 is coronal CT scan of a porcine subject after an infusion of thermoembolic materials into a liver of the subject. FIG 13 is a photograph of a coronal cross-section of a porcine liver showing the effects of a local ablation of a targeted portion of the liver using the thermoembolization techniques described herein.

Like reference symbols in the various drawings indicate like elements.

### DETAILED DESCRIPTION OF ILLUSTRATIVE EMBODIMENTS

The thermoembolization devices and techniques described herein can be used to treat solid tumors that arise in number of circumstances, including liver cancer, lung cancer, renal cancer, bone cancer, kidney cancer, breast cancer, prostate cancer, sarcomas, or the like. These techniques may be useful, for example, to treat patients who are not surgical candidates due to the nature of the tumors or other intervening factors. For example, some patients with HCC or other types of liver cancer are not candidates for surgery. The thermoembolization systems described herein may be effective in the treatment of such liver cancer in a manner that is relatively convenient to the patient (e.g., possibly reducing the number of treatment sessions) and relatively cost-effective for the medical care provider (e.g., not necessarily requiring high-cost equipment such as RF ablation probes or the like). The thermoembolization techniques described herein are not limited to use in human patients. For example, the ablation systems described herein may be used to treat other animal patients, including mammalian patients.

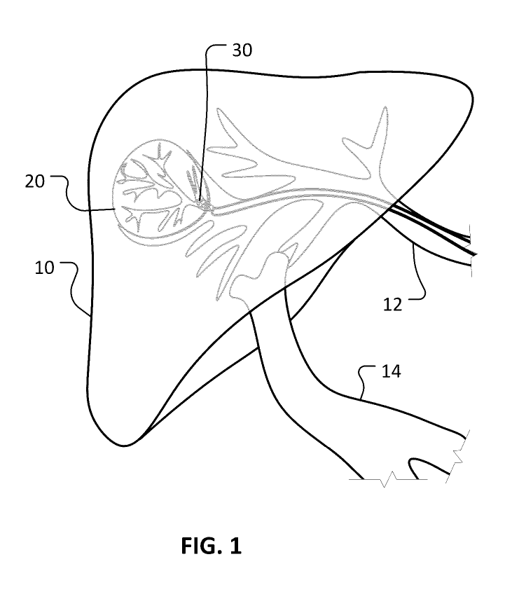

Referring to FIG. 1, in some cases a human liver 10 can include an example tumor 20. Oxygenated blood flows into the liver 10 from a hepatic artery 12. Nutrient-rich blood flows into the liver 10 from a portal vein 14. The tumor 20 receives oxygenated blood from branches of the hepatic artery 12. In accordance with various embodiments described herein, a thermoembolic material 30 delivered (via a lumen of a delivery device) through the hepatic artery 12 and into one or more branch vessels that feed the tumor 20. The thermoembolic material 30 is a material that is configured to stop flowing and stagnate in the branch vessels, in the portion of the hepatic artery 12 that feeds into the tumor, or a combination thereof. Because the supply of oxygenated blood to the tumor 20 from the hepatic artery is cut off, ischemia of the tumor 20 will result. Preferably, the thermoembolic material 30 is configured to induce simultaneous thermal and hypoxic stresses resulting from the thermoembolization technique described herein, which overwhelms tumor cells in a rapid manner prior to adaptation of the tumor 20.

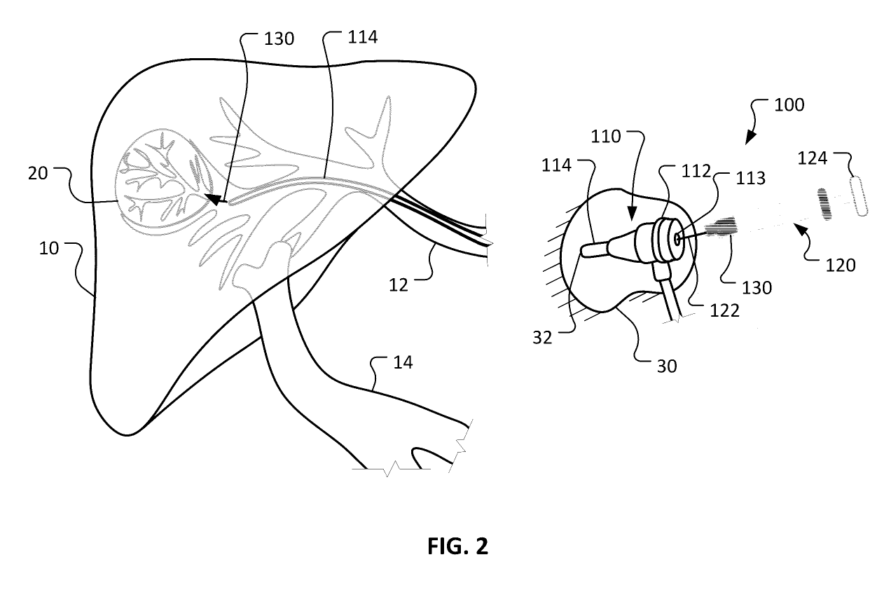

Referring to FIG. 2, the example tumor 20 in liver 10 can be treated using a thermoembolization system 100 to implement the thermoembolization techniques provided herein. In this example, the example thermoembolization system 100 includes a catheter 110 and a syringe 120 containing a reactive thermoembolic material 130. The thermoembolic material 130 can be injected into the catheter 110 using the syringe 120, such that the thermoembolic material 130 will be infused to the tumor 20. It should be understood that other types of devices and arrangements of devices can also be used to infuse the thermoembolic material 130 to the tumor 20.

In the depicted embodiment, the catheter 110 is inserted percutaneously through the skin 30 of a patient at a puncture site 32. In some embodiments, the procedure involves gaining access to the hepatic artery 12 by puncturing the common femoral artery in the right groin. A guide wire may be inserted and used to access the abdominal aorta, through the celiac trunk and common hepatic artery, and finally into the branch of the proper hepatic artery 12 supplying the tumor 20. The catheter 110 may be advanced over the guidewire, and then the guidewire can be removed. In some embodiments, this minimally invasive procedure is performed using x-ray fluoroscopy and/or other types of imaging modalities. While the thermoembolization techniques are described herein as being percutaneously delivered treatments, they also may be delivered as a treatment during open surgery, for example, as a method of intra-operative thermoembolization.

In the depicted embodiment, the catheter 110 includes a hub 112 and a catheter shaft 114. The catheter shaft 114 is attached to and extends distally from the hub 112. The hub 112 is configured to receive a needle 122 of the syringe 120. For example, in some embodiments the hub 112 includes a septum 113 through which the needle 112 can be inserted. When the needle 112 is inserted into the hub 112, a clinician can depress a plunger 124 of the syringe 120 to cause the thermoembolic material 130 to be injected into the catheter 110.

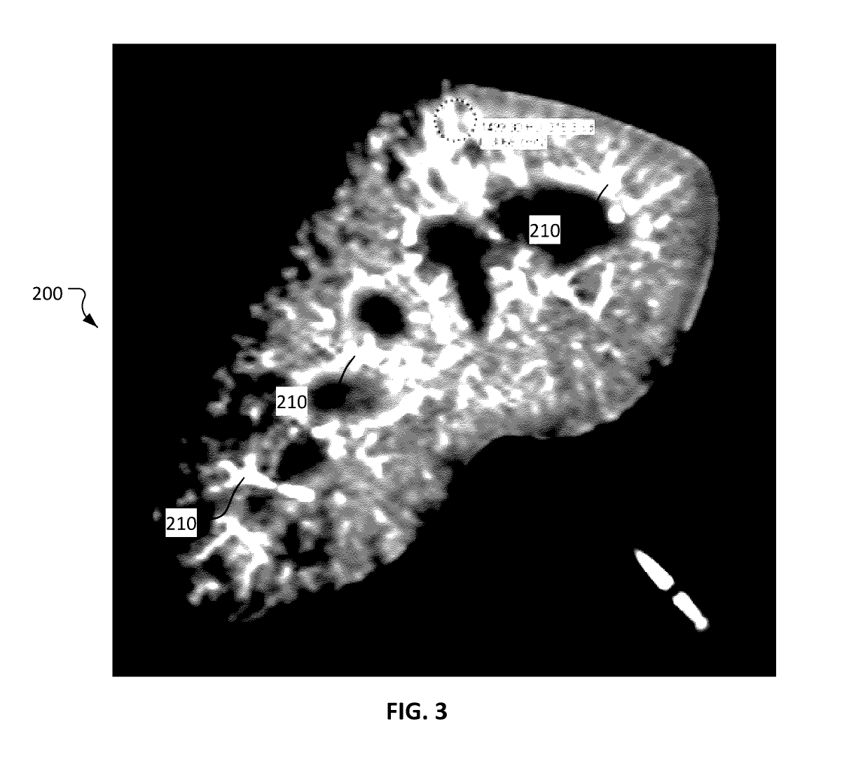

When the thermoembolic material 130 is injected into the catheter 110, the thermoembolic material 130 will flow through the catheter shaft 114 to the tumor 20. The thermoembolic material 130 will enter the branch vessel(s) that feed the tumor 20. Because of the small size of the branch vessel(s) that feed the tumor 20, the thermoembolic material 130 will stop flowing and stagnate in the branch vessel(s). In that manner, the thermoembolic material 130 causes an arterial embolization of the tumor 20, and ischemia of the tumor 20 will result. For example, FIG. 3 shows a CT scan of a kidney 200 that has received an infusion of thermoembolic material 130. In this example, the kidney 200 can be representative of a tumor such as, but not limited to, tumor 20. The white regions 210 show where the thermoembolic material 130 has stopped flowing and is dwelling in the microvasculature of the kidney 200. Hence, FIG. 3 illustrates that the thermoembolic material 130 arterially infused into the feeding vessels of a tissue structure (such as the kidney 200) will cause embolization of the tissue structure. In other words, this example using the kidney 200 simulates how an arterial infusion of the thermoembolic material 130 can cause embolization within a tumor.

Still referring to FIG. 2, in addition to the ischemia of the tumor 20 caused by the thermoembolic material 130, the thermoembolic material 130 includes one or more substances that exothermically reacts when the thermoembolic material 130 is within the branch vessel(s) that feed the tumor 20. The heat released by the exothermic reaction will cause an ablation of the tumor 20. Hence, the thermoembolic material 130 simultaneously causes embolization and hyperthermal ablation of the tumor 20.

In addition to simultaneous ischemia (hypoxia) and thermal stress, changes in pH of tissue may also be induced. The change in pH also can cause protein denaturation (leading to ablation by itself), and, depending on the material used, can either lower the pH (acidic, as in the acid chloride examples described herein) or raise the pH (alkaline or basic, such as with calcium oxide forming calcium hydroxide).

The thermoembolic material 130 can include various substances. In one example, the thermoembolic material 130 is comprised of an ethiodized oil contrast agent (such as LIPIODOL® and ETHIODOL®) and one or more reactive ingredients are dissolved, suspended, or emulsified in the ethiodized oil. LIPIODOL® (a type of ethiodized oil or oil-based contrast agent), is a poppyseed oil that can be used as a radio-opaque contrast agent to outline structures in radiological investigations. Ethiodized oil is composed of iodine combined with ethyl esters of fatty acids of poppyseed oil, primarily as ethyl monoiodostearate and ethyl diiodostearate. Ethiodized oils such as LIPIODOL® have a viscosity that causes it to stop flowing in small vessels, thereby occluding microcirculation vessels such as arterioles and/or capillaries.

The thermoembolic material 130 also optionally includes a reactive ingredient that causes an in situ exothermic reaction. In some such embodiments, the reactive ingredient reacts with water that is present in tissues (such as the tissue of the tumor 20). The heat of hydration for compounds corresponds to the heat that is released by hydration of one mole of ions at a constant pressure. The more the ion is hydrated, the more heat is released. The degree of hydration depends on the size and charge of the ion—the smaller the ion and the greater its charge, the more hydrated it will become, producing more heat. Thus, in some embodiments, a system can comprise a highly reactive reagent that, when it comes into contact with water present at the target tissue (or water that is added with the ablation reagent, e.g., via a dual chamber device), will undergo hydration or reaction, resulting in a release of heat.

The chemical agents of thermoembolic material 130 that can be used to generate heat of hydration include, without limitation, calcium oxide (CaO), which can be hydrated to calcium hydroxide (Ca(OH2)), and sulfuric acid (H2SO4). The hydration reaction of sulfuric acid is highly exothermic, and results in formation of sulfate and hydronium ions:

H2SO4+2H2O→2H3O++SO4−2

Other useful reagents for hydration reactions include, without limitation, potassium hydroxide (KOH) and sodium hydroxide (NaOH), hydration of which is quite exothermic.

Those skilled in the art will appreciate that some reagents are not likely to be suitable for the methods and systems provided herein. For example, hydration of some reagents may be more powerful than would be useful in an in vivo thermoembolization system.

When administered in liquid form, the reagent of thermoembolic material 130 to be hydrated can be provided at any suitable concentration, up to limits of solubility and/or availability (e.g., about 0.1 M, about 0.2 M, about 0.5 M, about 0.75 M, about 1 M, about 1.5 M, about 2 M, about 3 M, about 4 M, about 5 M, about 6 M, about 7 M, about 8 M, about 9 M, about 10 M, about 12 M, about 15 M, about 18 M, about 20 M, or any range therebetween, such as about 0.1 M to about 1 M, about 0.5 M to about 5 M, about 1 M to about 10 M, or about 17 M to about 19 M). Further, the reagent can be administered in any suitable amount (e.g., about 100 μl, about 250 μl, about 500 μl, about 750 μl, about 1 ml, about 2 ml, about 3 ml, about 4 ml, about 5 ml, about 6 ml, about 7 ml, about 8 ml, about 9 ml, about 10 ml, or any range therebetween, such as about 100 μl to about 1 ml, about 500 μl to about 5 ml, or about 1 ml to about 10 ml).

In some embodiments, a reagent to be hydrated may be administered in thermoembolic material 130 as a gel or a solid. For example, solid pieces of CaO (e.g., as a rod, a bead, or any other suitable form) in suspension in the thermoembolic material 130 can be infused to a target tissue (e.g., tumor 20) to be treated by thermoembolization. In some embodiments, such beads are about 100 μm in diameter. Moreover, in some embodiments such beads may be made of a core of CaO that is coated with an inert material that gradually dissolves in situ (e.g., over a period of about 24 hours). As the inert material coating dissolves, hydration of the CaO can occur such that heat for abating the tumor 20 is released. In addition, it is noted that in some cases, hydration can result in products (e.g., Ca(OH)2) that may be therapeutically beneficial by, for example, sensitizing cells to the heat of hydration by altering among other things the pH of the local environment. Such products may themselves also be useful for cell kill due to intrinsic properties such as but not limited to an extreme of pH.

In some embodiments, the thermoembolic material 130 may include other reactive substances. For example, the thermoembolic material 130 may provide useful imaging or other analyzable features (e.g., fluorescence, nuclear isotopes, MR imaging characteristics, or the like) to permit a health care professional to evaluate the distribution of the thermoembolic material 130 in the targeted tissue.

In some embodiments, the thermoembolic material 130 may include a denaturing agent that enhances the tissue ablation process. A denaturing agent as described herein can be mixed with the thermoembolic material 130 prior to delivery to a tumor site. The denaturing agent may act upon the targeted tissue to enhance the ablation effects caused by the thermochemical hydration reaction.

Moreover, in some embodiments, a drug may be added to the thermoembolic material 130, so as to provide a pharmacological effect on the targeted tissue in addition to the thermoembolization effects. In one example, a chemotherapy drug can be added to a delivery device to mix with the thermoembolic material 130 prior to injection. The chemotherapy drug can be administered to the targeted tissue to provide pharmacological effects contemporaneously with the ablation and embolization effects from the thermoembolic material 130. In another example, an anesthetic (e.g., lidocaine or procaine) can be administered to the targeted tissue to assist with pain control.

Additional examples of reactive electrophilic reagents include, but are not limited to, silica gel with adsorbed sodium (e.g., materials produced by SiGNa Chemistry, Inc. of New York, N.Y.), acetic anhydride, ethyl chloroformate, malonyl chloride, acetyl chloride, acetic anhydride, acetyl chloride, acetyl bromide, carboxylic acid anhydrides, other anhydrides, other acid halides, sulfonyl or phosphonyl anhydrides and halides (such as SOCl2, POCl3, PCl3, etc.), inorganic acid halides, chloroformates, chlorides, acetic anhydride, ethyl chloroformate, malonyl chloride, acetyl chloride, and the like.

In some embodiments, the electrophile of the thermoembolic material 130 can react with nucleophiles present at the treatment site to produce heat and increase local temperature. For example, the nucleophiles present at the treatment site may either be weak nucleophiles inherent in the targeted tissue area (e.g., protein or carbohydrate molecules disposed on cell surfaces or the like), nucleophiles deposited at the treatment site (generally prior to electrophile injection), or a combination thereof. In addition to the substantial reaction heat generated locally at the targeted tissue site when the electrophile is injected (to ablate the targeted tissue), such ablation techniques can also provide a denaturing effect in which a localized residual acidic or basic/alcohol environment operates to inhibit tumor growth or cell production in the local area for a period of time after the injection of the thermoembolic material 130. In these circumstances, the thermoembolic material 130 injection can generate significant ablation heat while still being a relatively small dosage, thereby reducing the overall acid load applied to the patient's system. Accordingly, the thermoembolization techniques described herein may permit a physician to treat multiple target locations in a single session.

Some of the techniques described herein may permit a health care professional (e.g., a physician) to simultaneously infuse at least two thermoembolization reagents without mixing the reagents until the reagents reach the distal portion of the delivery catheter shaft 114. In some embodiments, the catheter shaft 114 can be a dual lumen catheter that isolates the two thermoembolization reagents from each other until the reagents have exited the catheter shaft 114, or until the two thermoembolization reagents are about to exit the catheter shaft 114.

Other techniques for thermoembolization of tumor tissue may include chemical ablation by denaturation and/or inducement of cell death (e.g., via apoptosis). These methods may include administration of one, two, or more chemical ablation reagents. When multiple reagents are used, they may be administered simultaneously, and may be mixed prior to being taken up in the delivery cannula, or upon reaching the distal portion of the cannula. Such ablation techniques may provide a solution with a limited and safe level of reagents.

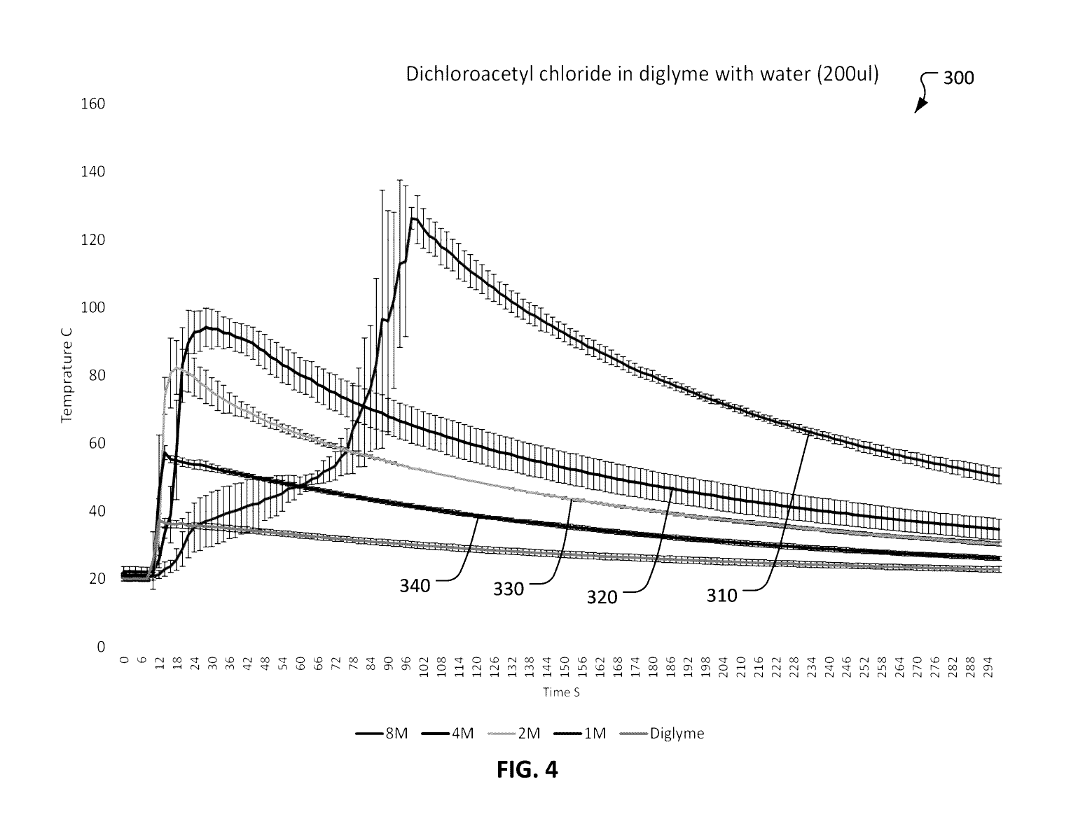

Referring to FIG. 4, it can be shown that acid chlorides dissolved in a solvent exothermically react with water as illustrated by temperature plot graph 300. In this empirical example, the acid chloride used was dichloroacetyl chloride, and the solvent used was diglyme. 200 μl of water was trickled into the solution of dichloroacetyl chloride and diglyme. Temperature plot line 310 shows the resulting temperature rise of an eight (8) molar solution of dichloroacetyl chloride in diglyme. Temperature plot line 320 shows the resulting temperature rise of a four (4) molar solution of dichloroacetyl chloride in diglyme. Temperature plot line 330 shows the resulting temperature rise of a two (2) molar solution of dichloroacetyl chloride in diglyme. Temperature plot line 340 shows the resulting temperature rise of a one (1) molar solution of dichloroacetyl chloride in diglyme. The temperature plot graph 300 illustrates that a substantial amount of heat is released by such an exothermic reaction. Such released heat can be advantageously used for the thermoembolization techniques provided herein.

A number of embodiments of the invention have been described. Nevertheless, it will be understood that various modifications may be made without departing from the scope of the invention. Accordingly, other embodiments are within the scope of the claims herein.

### EXAMPLES

### Thermoembolization of Porcine Kidney

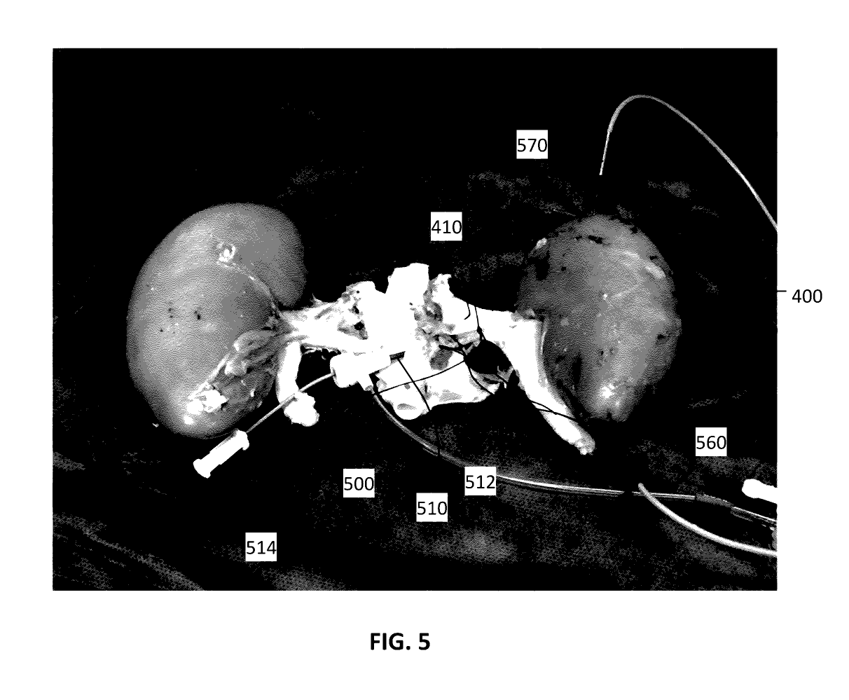

Referring to FIG. 5, a thermoembolization proof of concept experiment was conducted using porcine kidneys as the target tissue. The experimental set up included a porcine kidney 400, a catheter system 500, a first temperature sensor 560, and a second temperature sensor 570. In this example, the porcine kidney 400 was used as a proxy for a tumor that can be treated using the thermoembolization systems and techniques provided herein.

In this example, the catheter system 500 included a hub 510, a catheter shaft 512, and an obturator 514. The obturator 514 was used to reduce the dead space within the catheter system 500, (i.e., so that less thermoembolic material was needed). The thermoembolic material was injected into the proximal hub of the obturator 514 such that the material flowed through the obturator 514 and into the catheter shaft 512. The catheter shaft 512 of the catheter system 500 was inserted into a renal artery 420 leading to the porcine kidney 400. About 5 to 10 ml of thermoembolic material was injected.

The thermoembolic material used in this example was a four (4) molar solution of dichloroacetyl chloride in mineral oil. The temperature of the kidney 400 was monitored during and after the infusion of the thermoembolic material using the first temperature sensor 560 and the second temperature sensor 570.

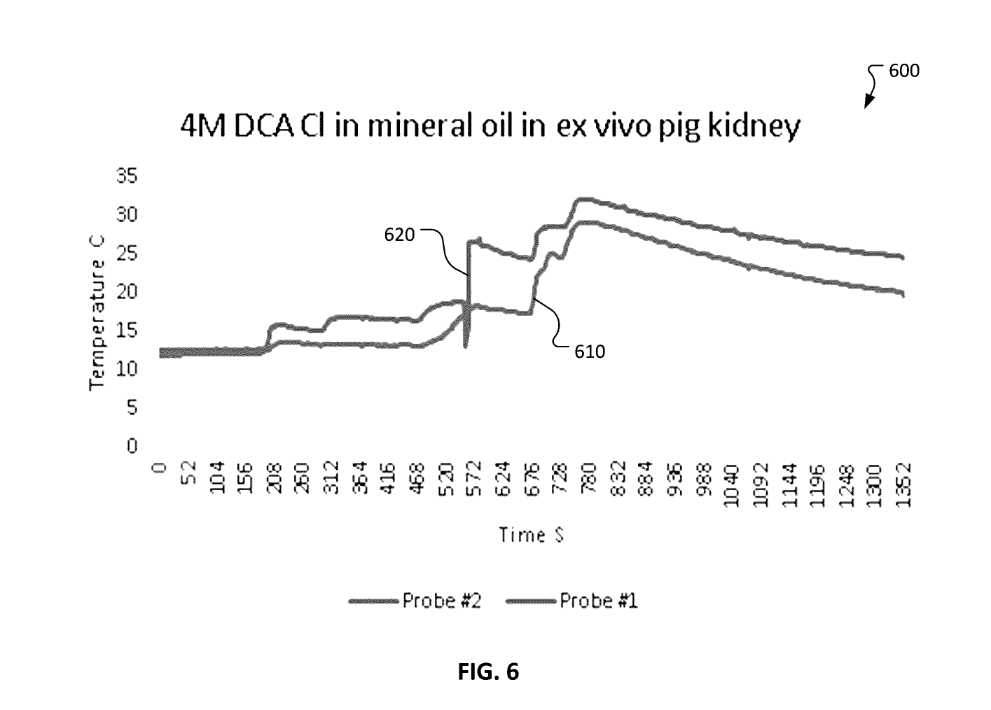

Referring also to FIG. 6, temperature plots 610 and 620 versus time for the readings from the first temperature sensor 560 and the second temperature sensor 570 are shown in graph 600. Each of the temperature sensors 560 and 570 detected an increase in temperature from the exothermic reaction of about 20° C. Such a temperature rise is sufficient for ablation the tissue. Hence, this experiment successfully modeled the thermoembolization devices and techniques provided herein.

In some cases, in vivo temperature measurements of tissues receiving the thermoembolization treatment described herein can be acquired using fluoroptic thermal probes. That is, for example while magnetic resonance (MR) imaging is being used to visualize the target tissue, one or more fluoroptic thermal probes can be used to measure the temperature of the target tissue while in a magnetic field from the MR imaging.

### In Vivo Thermoembolization of Porcine Liver

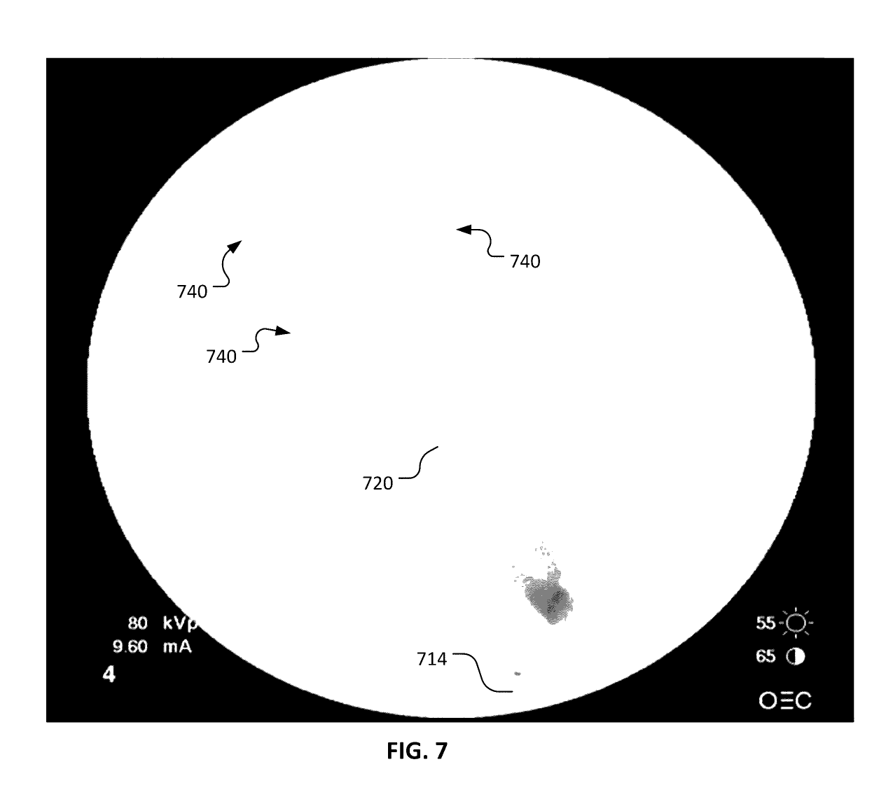

FIGS. 7-10 are a series of fluoroscopic images (e.g., angiography) of a porcine subject that is undergoing a liver catheterization and thermoembolization infusion treatment using a micro-catheter. The images show how a particular branch of a hepatic artery can be strategically selected and accessed by the micro-catheter. Accordingly, a targeted tissue (e.g., tumor tissue) can receive a thermoembolization treatment while non-targeted surrounding tissues can be left substantially untreated. This concept is also referred to herein as “selectivity.” While the example here is provided in the context of a liver, it should be understood that other target tissues including tumor tissues can be selectively treated in an analogous manner.

Referring to FIG. 7, a catheter 714 is approaching a liver from an aorta of the subject. A white radiopaque marker near the tip of catheter 714 can be readily visualized in these fluoroscopic images. A common hepatic artery 720 is visible. Catheter 714 can be routed into common hepatic artery 720. A branch structure 740 extends from common hepatic artery 720. As described further below, catheter 714 can also be routed into particular branch vessels of branch structure 740 to treat a particular area of targeted tissue.

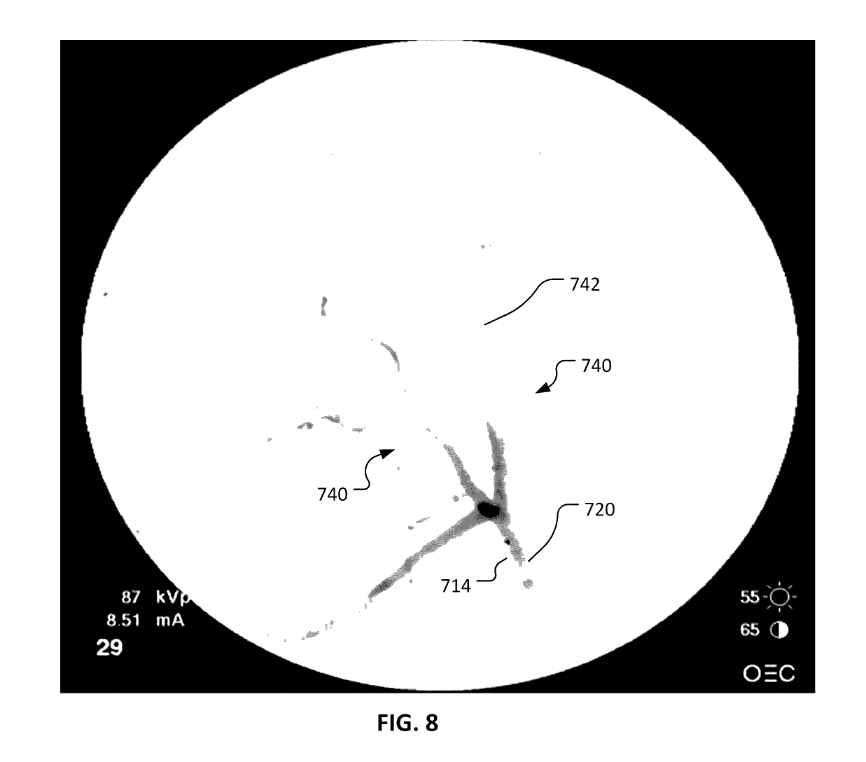

Referring to FIG. 8, the tip of catheter 714 has been advanced close to where common hepatic artery 720 divides into branch structure 740. From here, micro-catheter 714 can be selectively advanced into one or more particular branches of branch structure 740 so that a particular targeted portion of the tissue can be treated. This selectivity illustrates how, for example, a tumor growth within a liver can be treated using the thermoembolization treatment techniques while leaving healthy portions of the liver substantially untreated. For example, tissue supplied by a branch vessel 742 can be selected as the target tissue, and catheter 714 can be advanced into branch vessel 742 accordingly. Hence, it can been seen that catheter 714 can be advanced quite deeply into branch structure 740 to selectively treat local areas of tissue.

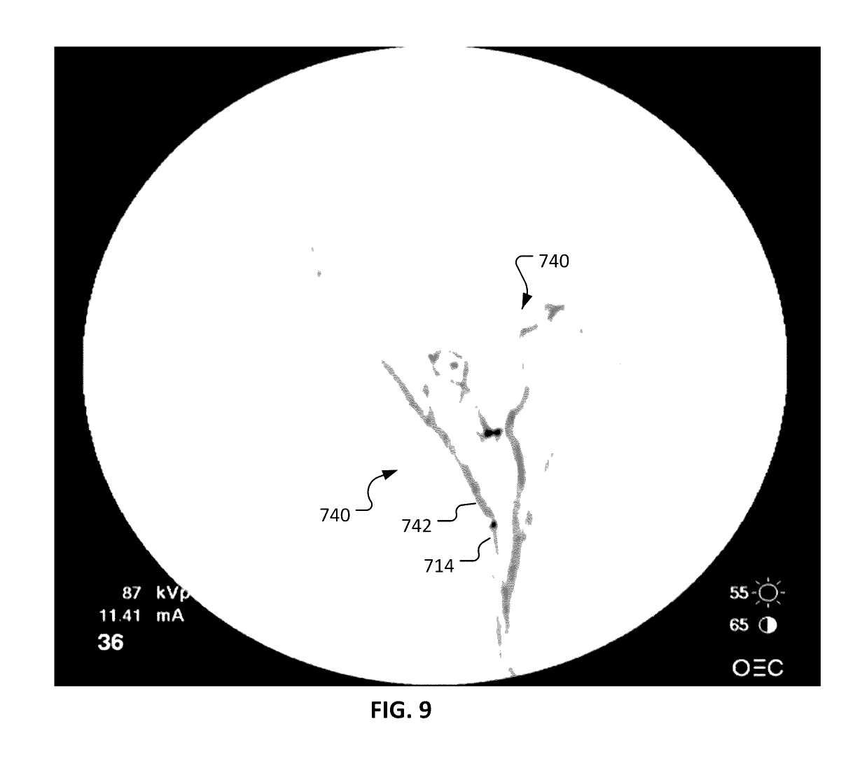

Referring to FIG. 9, the tip of catheter 714 has been advanced into branch vessel 742. While branch vessel 742 has been selected in this example, it should be understood that one or more of any of the branch vessels of branch structure 740 can be selected in correspondence with the targeted tissue to be selectively treated.

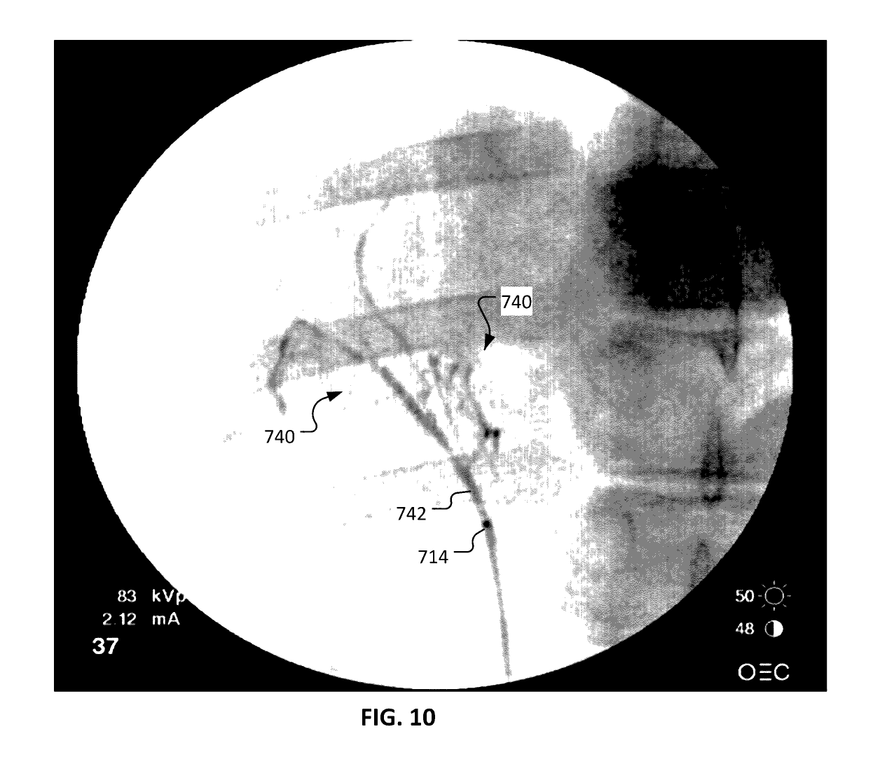

Referring to FIG. 10, with the tip of catheter 714 in branch vessel 742, a thermoembolization infusion treatment can be performed as shown. In this case, thermoembolic material comprising LIPIODOL® was infused. Preferably, the thermoembolic material is configured to induce simultaneous thermal and hypoxic stresses resulting from the thermoembolization technique described herein, which overwhelms tumor cells in a rapid manner prior to adaptation of the tumor.

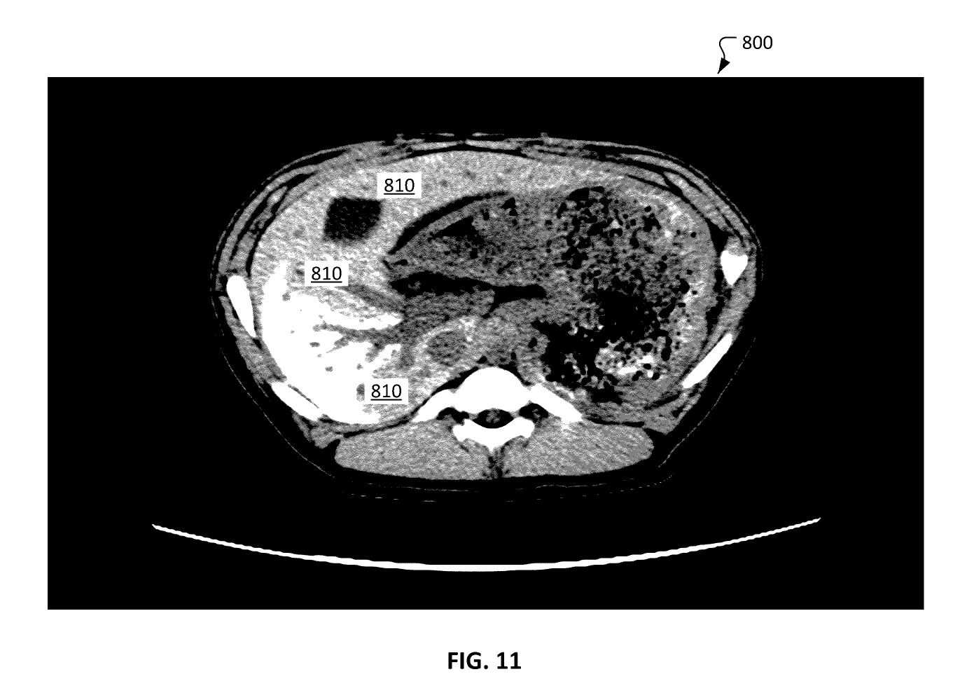

Referring to FIG. 11, a CT image 800 of an axial cross-section of a porcine subject shows a crescent-shaped cross-section of a liver 810 of which a portion that has received a thermoembolization infusion treatment in accordance with the devices and techniques described herein. The portal vein of liver 810 (the branched area within liver 810) can be visualized in image 800. No reactive thermoembolic material was injected into the portal vein in this case. Rather, one or more branches of the portal vein received such injections.

The targeted tissue (which can be seen in image 800 as the light-colored portion of liver 810) is receiving thermoembolization treatment, while the remaining portions of liver 810 are not being treated. The targeted tissue in this example was less than about 20% of the subject's liver 810. This exemplifies the selectivity of the thermoembolization devices and techniques described herein.

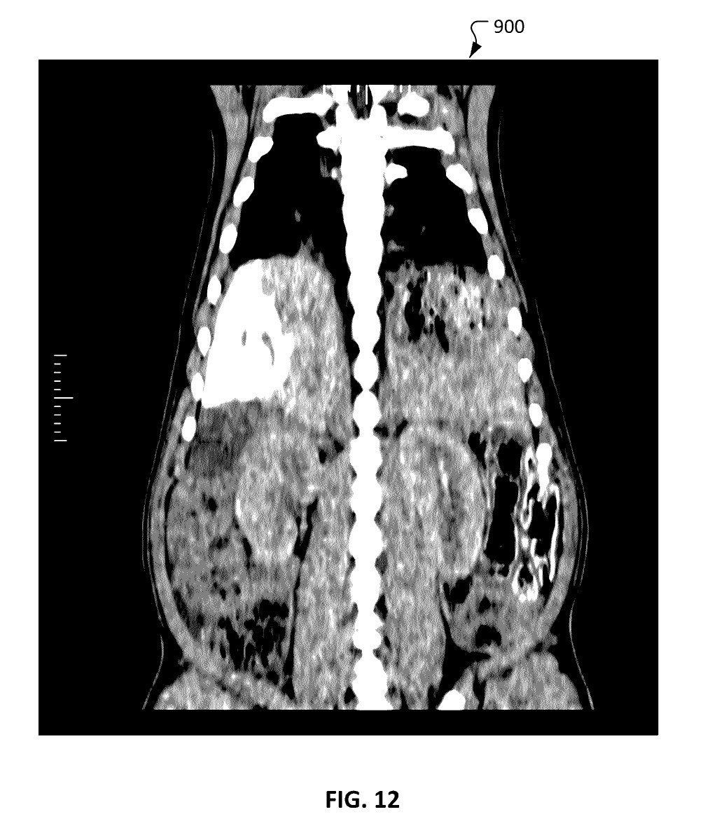

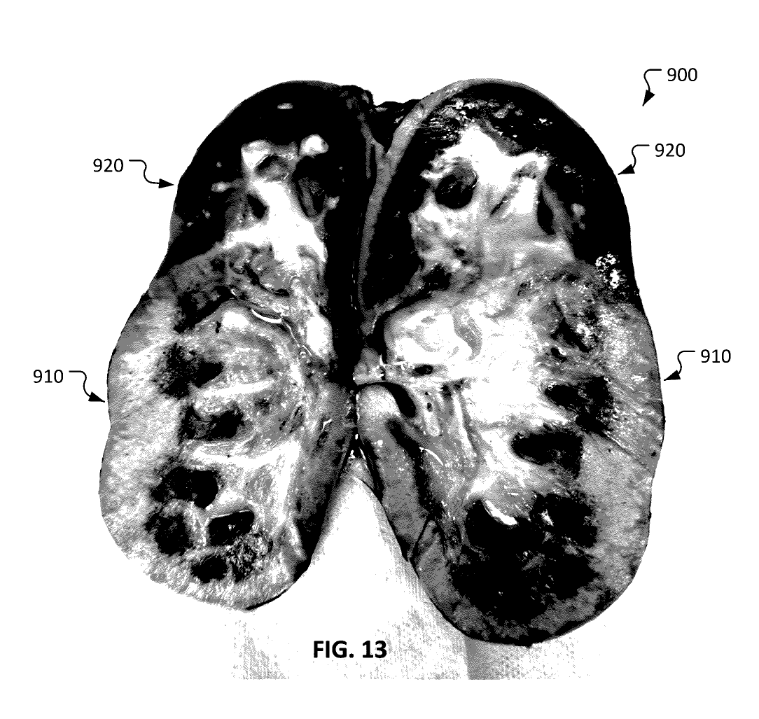

Referring to FIG. 12, a CT image 900 is a coronal CT scan showing a porcine subject after an infusion of thermoembolic materials into a liver of the subject. Again, here just a targeted portion of tissue (the light-colored area) is receiving the thermoembolization infusion treatment using the devices and techniques described herein.

Referring to FIG. 13, a photograph showing a coronal cross-sectional dissection of a partially-treated porcine liver 900 shows the effects of a local ablation of a targeted portion of tissue using the thermoembolization techniques described herein. That is, liver 900 includes an ablated portion 910 and an untreated portion 920. Here again, this experimental example illustrates the aspect of target tissue selectivity in regard to the thermoembolization infusion treatments using the devices and techniques described herein.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A thermoembolization system for treating a tissue, the thermoembolization system comprising:
a percutaneous liquid delivery catheter defining at least one lumen extending from a proximal portion of the percutaneous liquid delivery catheter to a distal portion of the percutaneous liquid delivery catheter, the distal portion of the percutaneous liquid delivery catheter configured for insertion into an arterial vessel such that a distal tip of the distal portion of the percutaneous liquid delivery catheter is positionable adjacent the tissue;
a liquid dispensing device including a reservoir and a portion that is configured to releasably couple with the proximal portion of the percutaneous liquid delivery cannula such that the liquid dispensing device can inject a liquid from the reservoir into the lumen of the percutaneous liquid delivery catheter; and
a thermoembolic liquid contained within the reservoir, wherein the thermoembolic liquid has a viscosity sufficient to cause an occlusion of a microvasculature of the tissue, and wherein the thermoembolic material includes a reagent that exothermically reacts with a substance.

2. The thermoembolization system of claim 1, wherein the reagent comprises calcium oxide.

3. The thermoembolization system of claim 2, wherein the substance is water.

4. The thermoembolization system of claim 1, wherein the reagent comprises sulfuric acid.

5. The thermoembolization system of claim 1, wherein the thermoembolic liquid comprises an ethiodized oil.

6. The thermoembolization system of claim 1, wherein the liquid dispensing device is a syringe.

7. The thermoembolization system of claim 1, wherein the reagent comprises beads.

8. The thermoembolization system of claim 7, wherein the beads comprise calcium oxide.

9. The thermoembolization system of claim 8, wherein the beads comprise an inert coating surrounding the calcium oxide.

10. The thermoembolization system of claim 1, wherein the thermoembolic liquid is radiopaque.

11. A method of treating a tumor, the method comprising infusing a thermoembolic liquid into an arterial vessel feeding the tumor, wherein the thermoembolic liquid has a viscosity sufficient to cause an occlusion of a microvasculature of the tumor, and wherein the thermoembolic liquid includes a reagent that exothermically reacts with a substance to generate heat sufficient to ablate at least a portion of the tumor while the thermoembolic liquid is occluding the microvasculature.

12. The method of claim 11, wherein the reagent comprises calcium oxide or sulfuric acid.

13. The method of claim 12, wherein the substance is water present in the tumor.

14. The method of claim 11, wherein the thermoembolic liquid comprises an ethiodized oil.

15. The method of claim 11, wherein the reagent comprises beads.

16. The method of claim 15, wherein the beads comprise calcium oxide.

17. The method of claim 16, wherein the beads comprise an inert coating surrounding the calcium oxide.

18. The method of claim 11, wherein the thermoembolic liquid is radiopaque.

19. The method of claim 11, wherein the tumor is located in a human liver.

20. The method of claim 11, wherein the tumor is located in at least one of a kidney, a bone, or a prostate.
